(19) United States
(12) Patent Application Publication
(10) Pub. No.: US 2002/0119562 A1
(43) Pub. Date: Aug. 29, 2002

(54) Method for selecting improved vectors

(76) Inventors: Alan John Kingsman, Oxford (GB); Jason Slingsby, London (GB); Melvyn Yap, London (GB)

Correspondence Address: FROMMER LAWRENCE & HAUG, 745 FIFTH AVENUE- 10TH FL., NEW YORK, NY, 10151, US

(21) Appl. No.: 10/002,598

(22) Filed: Nov. 15, 2001

Related U.S. Application Data
(63) Continuation-in-part of application No. PCT/GB00/01964, filed on May 22, 2000.

(30) Foreign Application Priority Data
May 21, 1999 (GB) GB 9911963.8
May 24, 1999 (GB) GB 9911975.2

(51) Int. Cl. C12N 7/00; C12N 5/06; C07H 21/02; C07H 21/04; A61K 39/12; A61K 39/21; C12N 7/01; C12N 15/00; C12N 15/09; C12N 15/63; C12N 15/70; C12N 15/74; C12N 5/00; C12N 5/02

(52) U.S. Cl. 435/320.1; 435/235.1; 435/325; 536/23.72; 424/199.1; 424/207.1

(57) ABSTRACT

A method is provided for selecting an improved retroviral genome having an improved packaging efficiency which method comprises: a) introducing one or more random mutations into a retroviral genome comprising a packaging signal; b) introducing the mutagenised retroviral genome into a host cell expressing viral polypeptides required for packaging the retroviral genome; c) determining whether retroviral packaging efficiency in the cell is improved as compared with a retroviral genome comprising a non-mutated packaging signal; d) selecting a viral genome which has improved packaging efficiency.

### FIELD OF THE INVENTION

The present invention relates to a method for improving the packaging efficiency of retroviral vectors.

### BACKGROUND TO THE INVENTION

Retroviral vectors are now widely used as vehicles to deliver genes into cells. Their popularity stems from the fact that they are safe, easy to produce and mediate stable integration of the gene that they carry into the genome of the target cell. This enables long-term expression of the delivered gene (Miller. 1997).

The production of retroviruses is far from efficient. Typically, less than ten percent of viral particles in a retroviral stock is infectious because the rest of the particles do not carry a viral genome. In the wild type virus, the gag/gag-pol gene products are translated from the fill-length RNA transcript, which is also packaged. One hypothesis for the poor packaging efficiency lies in the competition between the processes of packaging and the translation (Sonstegard and Hackett 1996). Tis is due to the signal for packaging being located very close to the ribosomal binding site. Binding of the gag protein to the packaging signal obstructs the binding of ribosomes and thus inhibits its own translation. During the course of evolution, a stronger packaging signal was not selected because it will bind more strongly to the gag protein and limit its production.

In the production of retroviral vectors, however, the gag/gag-pol and genome components have been separated into different expression cassettes (Soneoka et al., 1995). The processes of packaging and translation have therefore been uncoupled. Hence, it is theoretically possible that a viral genome could be developed with high packaging efficiency without compromising the production of gag/gag-pol.

Previous attempts in obtaining improved novel retroviral sequences have either been through serial passage of the replication competent virus (Taplitz and Coffin. 1997) or by in vitro selection procedures (Allen et al., 1996; Berglund et al., 1997). Serial passage of the virus takes a long time. As for in vitro selection, there are many parameters that need to be optimised. These include the conditions required to perform mutagenesis and selection. Furthermore it often relies on the size of library of random mutations, which in turn depends on the successful cloning of the mutagenised DNA.

Furthermore, none of these previous attempts to produce novel retroviral sequences have focussed on trying to improve retroviral packaging beyond its natural efficiency.

### SUMMARY OF THE INVENTION

We have found, while investigating effects of the stoichiometry of viral components on retroviral production using a transient transfection system, that it is possible to produce a viral stock in which substantially all the particles are infectious by ensuring that they are all filled with genomic RNA. Our results show that this may be achieved by using a lower proportion of gag/gag-pol construct compared with env and genome.

Accordingly, the present invention provides a method for enhancing the efficiency of retroviral packaging which method comprises expressing in a producer cell at least a first nucleotide sequence encoding a retroviral gag-pol polypeptide, a second nucleotide sequence encoding a retroviral envelope polypeptide and a third nucleotide sequence encoding a retroviral genome wherein the ratio of the first nucleotide sequence to the second and third nucleotide sequence is x:y:z wherein x is less than 1 and y and z are 1. In other words, the ratio of the first nucleotide sequence to the second and third nucleotide sequence is less than 1:1:1.

Preferably, the ratio of the first nucleotide sequence encoding a retroviral gag-pol polypeptide to the second and third nucleotide is less than 0.8:1:1 or 0.5:1:1, more preferably less than 0.2:1:1.

Preferably the proportion of the number of infectious retroviral particles to the total number of retroviral particles produced is greater than 15%, more preferably greater than 25% or 50%.

The present invention also provides a composition comprising retroviral particles produced according to the method of the invention described above, and a producer cell which has enhanced retroviral packaging efficiency due to the fact it expresses a lower proportion of gag/gag-pol construct compared with env and genome.

Another way to increase the proportion of infectious particles would be to improve the packaging efficiency of the genome by modifying the packaging signal. However, to date, there have only been attempts to identify naturally occurring viral packaging signals and incorporate these into retroviral vectors (Adam and Miller, 1988). Such vectors will, at best, only be packaged with wild type efficiencies.

We have therefore developed an in vivo strategy to select a vector genome with improved packaging efficiency which involves shuttling a retroviral vector between a mutagenic strain of Epicurian coli, where random mutations are introduced in its sequence, and mammalian cells, where selection is made for better packaging efficiency (FIG. 2).

Increasing the packaging efficiency results in viral stocks that contain more infectious particles. This leads to higher end-point titres. Furthermore, transduction efficiencies are also improved because there are fewer defective particles that will otherwise obstruct the binding of infectious particles to the target cell receptors.

The in vivo method of selection that we describe is rapid and easy to perform. It only involves extracting and introducing DNA from mammalian and bacteria cells. These are extremely well characterised and optimised processes.

Accordingly the present invention also provides a method for selecting an improved retroviral genome having an improved packaging efficiency which method comprises:

(a) introducing one or more random mutations into a retroviral genome comprising a packaging signal;

(b) introducing the mutagenised retroviral genome into a host cell expressing viral polypeptides required for packaging the retroviral genome;

(c) determining whether retroviral packaging efficiency in the cell is improved as compared with a retroviral genome comprising a non-mutated packaging signal;

(d) selecting a viral genome which has improved packaging efficiency.

Preferably the method comprises an additional step (e) of determining the sequence of all or part of the viral genome to identify the sequence of the packaging signal.

Preferably step (a) is carried out in a bacterial strain which introduces random mutations into the retroviral genome. A particularly preferred bacterial strain is a mutagenic strain of Epicurian coli.

Preferably the host cell of steps (b) and (c) is a mammalian cell. In a particularly preferred embodiment, the host cell comprises at least:

(i) a first nucleotide sequence encoding a retroviral gag-pol polypeptide;

(ii) a second nucleotide sequence encoding a retroviral envelope polypeptide; and

(iii) a third nucleotide encoding a retroviral genome comprising a non-mutated packaging signal,

wherein the third nucleotide is present as part of a nucleic acid vector which lacks a selectable marker; the mutagenised retroviral genome is present as part of a nucleic acid vector which contains the selectable marker; and the ratio of the vector comprising the third nucleotide to the vector comprising the mutagenised retroviral genome is greater than 2:1, preferably greater than 5:1.

It is preferred that the packaging efficiency, measured as the proportion of the number of infectious retroviral particles to the total number of retroviral particles produced is greater than 25%, more preferably greater than 50%. The number of infectious retroviral particles in a viral stock (i.e. the titre) may be determined by techniques known in the art, for example by transducing mammalian cells. The total number of retroviral particles may be measured by, for example, reverse transcription assays.

The present invention also provides a retroviral genome obtained by the selection method of the invention. Preferably the retroviral genome has a packaging efficiency, measured as the proportion of the number of infectious retroviral particles to the total number of retroviral particles produced of greater than 25%, more preferably greater than 50%.

The retroviral genome's packaging signal may be cloned from the improved retroviral genome, or its sequence determined and used to synthesis a packaging signal for use in producing an improved retroviral genome. Thus the invention also provides a retroviral packaging signal obtainable from a retroviral genome selected by the method of the invention.

The present invention further provides a non-naturally occurring retroviral packaging signal, which as measured when part of a retroviral genome, has a packaging efficiency of at least 15%, preferably at least 20, 25, 35 or 50%.

In a further aspect the present invention provides a nucleic acid comprising a retroviral packing signal of the invention and a retroviral vector comprising a retroviral packing signal of the invention. The retroviral vector may be a lentiviral vector.

The present invention also provides a producer cell which comprises a retroviral genome, retroviral packaging signal or retroviral vector according to the invention.

The present invention also provides a composition comprising infectious retroviral particles produced according to the methods of the invention and/or using improved retroviral genomes obtained by the methods of the invention. Such compositions may, for example, be used in therapy.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al., Current Protocols in Molecular Biology (1995), John Wiley & Sons, Inc.

Retroviruses

The retroviral vectors used in the production of mutagenised retroviral genomes and infectious retroviruses according to the methods of the present invention may be derived from or may be derivable from any suitable retrovirus. A large number of different retroviruses have been identified. Examples include: murine leukemia virus (MLV), human immunodeficiency virus (HIV), simian immunodeficiency virus, human T-cell leukemia virus (HTLV). equine infectious anaemia virus (EIAV), mouse mammary tumour virus (MMTV). Rous sarcoma virus (RSV), Fujinami sarcoma virus (FuSV). Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “Retroviruses”. Cold Spring Harbour Laboratory Press Eds: J M Coffin, S M Hughes, H E Varmus pp 758-763.

Details on the genomic structure of some retroviruses may be found in the art. By way of example, details on HIV and Mo-MLV may be found from the NCBI Genbank (Genome Accession Nos. AF033819 and AF033811, respectively).

The lentivirus group can be split even further into “primate” and “non-primate”. Examples of primate lentiviruses include human immunodeficiency virus (HIV), the causative agent of human auto-immunodeficiency syndrome (AIDS), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells (Lewis et al1992 EMBO. J 11: 3053-3058; Lewis and Emerman 1994 J. Virol. 68: 510-516). In contrast, other retroviruses—such as MLV—are unable to infect non-dividing cells such as those that make up, for example, muscle, brain, lung and liver tissue.

Preferred vectors optimisable by the method of the present invention are recombinant retroviral vectors, in particular recombinant lentiviral vectors, in particular minimal lentiviral vectors, teachings relating to which are disclosed in WO 99/32646 and in WO98/17815.

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. More complex retroviruses have additional features. such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a defective retroviral vector genome gag, pol and env may be absent or not functional.

The R regions at both ends of the RNA are repeated sequences. U5 and U3 represent unique sequences at the 5′ and 3′ ends of the RNA genome respectively.

In a typical retroviral vector for use in gene therapy, at least part of one or more of the gag, pol and env protein coding regions essential for replication may be removed from the virus. This makes the retroviral vector replication-defective. The removed portions may even be replaced by a nucleotide sequence of interest (NOI), such as a nucleotide sequence encoding a therapeutic product, to generate a virus capable of integrating its genome into a host genome but wherein the modified viral genome is unable to propagate itself due to a lack of structural proteins. When integrated in the host genome, expression of the NOI occurs—resulting in, for example, a therapeutic and/or a diagnostic effect. Thus, the transfer of an NOI into a site of interest is typically achieved by: integrating the NOI into the recombinant viral vector; packaging the modified viral vector into a virion coat; and allowing transduction of a site of interest—such as a targeted cell or a targeted cell population.

A minimal retroviral genome for use in the present invention will therefore comprise (5′) R-U5—one or more first nucleotide sequences—U3-R (3′). However, the plasmid vector used to produce the retroviral genome within a host cell/packaging cell will also include transcriptional regulatory control sequences operably linked to the retroviral genome to direct transcription of the genome in a host cell/packaging cell. These regulatory sequences may be the natural sequences associated with the transcribed retroviral sequence, i.e. the 5′ U3 region, or they may be a heterologous promoter such as another viral promoter, for example the CMV promoter.

Some retroviral genomes require additional sequences for efficient virus production. For example, in the case of HIV, rev and RRE sequence are preferably included. However the requirement for rev and RRE can be reduced or eliminated by codon optimisation.

Codon optimisation causes to an improvement in codon usage. By way of example, alterations to the coding sequences for viral components may improve the sequences for codon usage in the mammalian cells or other cells which are to act as the producer cells for retroviral vector particle production. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

The retroviral vector may be produced using a codon optimised gag and a codon optimised pol or a codon optimised env.

Accessory genes encode variety of accessory proteins capable of modulating various aspects of retroviral replication and infectivity. These proteins are discussed in Coffin et al. Chapters 6 and 7. Examples of accessory proteins in lentiviral vectors include but are not limited to tat, rev, nef, vpr, vpu, vif, vpx. An example of a lentiviral vector useful in the present invention is one which has all of the accessory genes removed except rev.

Once the retroviral vector genome is integrated into the genome of its target cell as proviral DNA, the nucleotide sequences of interest need to be expressed. In a retrovirus, the promoter is located in the 5′ LTR U3 region of the provirus. In retroviral vectors, the promoter driving expression of a therapeutic gene may be the native retroviral promoter in the 5′ U3 region, or an alternative promoter engineered into the vector. The alternative promoter may physically replace the 5′ U3 promoter native to the retrovirus, or it may be incorporated at a different place within the vector genome such as between the LTRs.

Thus, an NOI will also be operably linked to a transcriptional regulatory control sequence to allow transcription of the NOI to occur in the target cell. The control sequence will typically be active in mammalian cells. The control sequence may, for example, be a viral promoter such as the natural viral promoter or a CMV promoter or it may be a mammalian promoter. It is particularly preferred to use a promoter that is preferentially active in a particular cell type or tissue type in which the virus to be treated primarily infects. Thus, in one embodiment, a tissue-specific regulatory sequences may be used. The regulatory control sequences driving expression of the one or more first nucleotide sequences may be constitutive or regulated promoters. Another particularly preferred regulatory construct comprises an hypoxia responsive element, such as is described in WO99/15684, the contents of which are incorporated herein by reference.

Replication-defective retroviral vectors are typically propagated, for example to prepare suitable titres of the retroviral vector for subsequent transduction, by using a combination of a packaging or helper cell line and the recombinant vector. That is to say, that the three packaging proteins can be provided in trans (see below).

Producer Cells

Retroviral producer cells are cells that contain all the elements necessary for the production of infectious recombinant retroviruses. These elements may be permanently present stably within the cell (for example integrated in the cell genome or in episomal form) and/or transiently provided, for example by transfection.

A packaging cell, by contrast, expresses one or more viral components required for packaging retroviraI DNA but lacks a psi region. Packaging cell lines typically comprise one or more of the retroviral gag, pol and env genes. Thus, the packaging cell line produces the structural proteins required for packaging retroviral DNA but it cannot brine about encapsidation due to the lack of a psi region. However, when a recombinant vector carrying a defective viral genome comprising a psi region and typically a nucleotide sequence of interest (NOI) is introduced into the packaging cell line, the helper proteins can package the psi-positive recombinant vector to produce the recombinant virus stock. This virus stock can be used to transduce cells to introduce the NOI into the genome of the target cells. It is preferred to use a psi packaging signal called psi plus, that contains additional sequences spanning from upstream of the splice donor to downstream of the gag start codon since this has been shown to increase viral titres.

The recombinant virus whose genome lacks all genes required to make viral proteins can tranduce only once and cannot propagate. These viral vectors which are only capable of a single round of transduction of target cells are known as replication defective vectors. Hence, the NOI is introduced into the host/target cell genome without the generation of potentially harmful retrovirus. A summary of the available packaging lines is presented in Coffin et al., 1997.

Packaging cell lines in which the gag, pol and env viral coding regions are carried on separate expression plasmid that are independently transfected into a packaging cell line are preferably used. This strategy, sometimes referred to as the three plasmid transfection method (Soneoka et al., 1995) reduces the potential for production of a replication-competent virus since three recombinant events are required for wild type viral production. As recombination is greatly facilitated by homology, reducing or eliminating homology between the genomes of the vector and the helper can also be used to reduce the problem of replication-competent helper virus production.

An alternative to stably transfected packaging cell lines is to use transiently transfected cell lines. Transient transfections may advantageously be used to measure levels of vector production when vectors are being developed. In this regard, transient transfection avoids the longer time required to generate stable vector-producing cell lines and may also be used if the vector or retroviral packaging components are toxic to cells. Components typically used to generate retroviral vectors include a plasmid encoding the gag/pol proteins a plasmid encoding the env protein and a plasmid containing an NOI. Vector production involves transient transfection of one or more of these components into cells containing the other required components. If the vector encodes toxic genes or genes that interfere with the replication of the host cell, such as inhibitors of the cell cycle or genes that induce apotosis, it may be difficult to generate stable vector-producing cell lines, but transient transfection can be used to produce the vector before the cells die. Also, cell lines have been developed using transient transfection that produce vector titre levels that are comparable to the levels obtained from stable vector-producing cell lines.

Producer cells can be produced either from packaging cells by introducing into the packaging cell any remaining viral components required for infectious retrovirus production or they can be produced by introduction into a non-packaging cell, such as a 293T cell, of all the components required for infectious retrovirus production.

Producer cells/packaging cells can be of any suitable cell type. Most commonly. mammalian producer cells are used but other cells, such as insect cells are not excluded. Clearly, the producer cells will need to be capable of efficiently translating the env and gag, pol mRNA. Many suitable producer/packaging cell lines are known in the art. The skilled person is also capable of making suitable packaging cell lines by, for example stably introducing a nucleotide construct encoding a packaging component into a cell line.

It is highly desirable to use high-titre virus preparations in both experimental and practical applications. Techniques for increasing viral titre include using a psi plus packaging signal as discussed above and concentration of viral stocks. In addition, the use of different envelope proteins, such as the G protein from vesicular-stomatitis virus has improved titres following concentration to 10⁹ per ml. However, typically the envelope protein will be chosen such that the viral particle will preferentially infect cells that are infected with the virus which it desired to treat. For example where an HIV vector is being used to treat HIV infection, the env protein used will be the HIV env protein.

Mutagenesis of Retroviral Genomes

In vitro evolution has gained much attention in recent times. It involves introducing random mutations in a DNA sequence followed by a selection procedure, thereby producing a gene with an improved or novel function (Joyce, 1992). The strength of the technology lies in the fact that there is no requirement to understand how a gene functions in order to change or improve it. All that is necessary is the generation of random mutations in the DNA sequence at a high frequency and a procedure to select the desired changes. There are several methods of generating random mutations. These include error-prone PCR (Beaudry and Joyce. 1992), DNA shuffling (Stemmer, 1994) and, more recently, transformation of a mutagenic strain of Epicurian coli (Bornscheuer et al., 1998). Other techniques are also known in the art for producing mutations (such as the use of ionising radiation or chemical mutagens). It is preferred in the context of the present invention to use a mutagenic bacterial strain such a mutagenic strain of Epicurian coli introduce mutations in a retroviral genome.

Generally, the retroviral genome will be present as part of a nucleic acid vector. Where mutagenesis is to take place in a host cell, in vivo, the nucleic acid vector will be chosen to be compatible with the host cell. It is particularly preferred to use a shuttle vector, i.e. a vector which can be propagated in more than one type of host (such as bacteria and mammalian cells). This enables mutagenesis to take place in, for example, a bacterial strain and then the mutagenised vector extracted and purified using standard techniques before introduction into the mammalian cell without the need for any cloning.

Thus the first stage is to subject the retroviral genome to mutagenesis. In a preferred embodiment this is achieved by transforming the retroviral genome into Epicurian coli cells such as XLl-Red cells (Stratagene). The pool of resulting mutants is then extracted using standard techniques such as large-scale plasmid isolation protocols.

Selection

Typically, a pool of mutagenised retroviral genomes are then introduced into host cells that are capable of packaging the genome (see above for details of packaging cells and producer cells). Competition for packaging is set up by co-transfecting the host cells with a non-mutagenised retroviral genome present as part of a vector which lacks a selectable marker suitable for use in the host cell. By contrast, the mutagenised retroviral genome is present as part of a vector which contains the selectable marker. Preferably, the amount of non-mutagenised genome is at least double, more preferably at least five times the amount of mutagenised genome.

Retroviral particles produced by the host cells are then used to transduce further cells, such as COS cells. Any resulting neomycin-resistant cells contain mutagenised retroviral genomes which are packaged more efficiently than the wild type retroviral genome. The In retroviral particles produced by these cells can be tested to determined their packing efficiency as described above.

Selected retroviral clones may be isolated and optionally subjected to additional mutagenesis/selection steps. Selected retroviral clones may also be isolated and their nucleotide sequence determined to establish the particular mutation or mutations which result in improved packaging efficiency. Typically these will be within the packing signal sequence.

The improved retroviral genomes may be used as the basis of constructing improved retroviral vectors, for example for use in delivering a therapeutic gene to a patient. In particular, the modified sequences may be cloned into existing retroviral vectors.

Uses

The application of the methods of the invention will enable the production of compositions comprising retroviral particles wherein a greater proportion are infectious than has been possible previously.

The infectious retroviral particles may comprise one or more coding sequences encoding therapeutic products. Therapeutic products include, but are not limited to. cytokines. hormones, antibodies, immunoglobulin fusion proteins, enzymes, immune co-stimulator molecules anti-sense RNA, a transdominant negative mutant of a target protein, a toxin. a conditional toxin, an antigen, a single chain antibody, tumour suppresser protein and growth factors. When included, such coding sequences are operatively linked to a suitable promoter.

Preferably the viral particles are combined with a pharmaceutically acceptable carrier or diluent to produce a pharmaceutical composition. Thus, the present invention also provides a pharmaceutical composition for treating an individual, wherein the composition comprises a therapeutically effective amount of the viral particle of the present invention. together with a pharmaceutically acceptable carrier, diluent, excipient or adjuvant. The pharmaceutical composition may be for human or animal usage.

The choice of pharmaceutical carrier, excipient or diluent can be selected with regard to the intended route of administration and standard pharmaceutical practice. Suitable carriers and diluents include isotonic saline solutions, for example phosphate-buffered saline. The pharmaceutical compositions may comprise as—or in addition to—the carrier, excipient or diluent any suitable binder(s), lubricant(s), suspending agent(s), coating agent(s). solubilising agent(s), and other carrier agents that may aid or increase the viral entry into the target site (such as for example a lipid delivery system).

The pharmaceutical composition may be formulated for parenteral, intramuscular. intravenous, intracranial, subcutaneous, intraocular or transdermal administration.

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously. intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The amount of virus administered is typically in the range of from 10³ to 10¹⁰ pfu. preferably from 10⁵ to 10⁸ pfu, more preferably from 10⁶ to 10⁷ pfu. When injected. typically 1-10 μl of virus in a pharmaceutically acceptable suitable carrier or diluent is administered.

Where the therapeutic sequence is under the control of an inducible regulatory sequence, it may only be necessary to induce gene expression for the duration of the treatment. Once the condition has been treated, the inducer is removed and expression of the NOI is stopped. This will clearly have clinical advantages. Such a system may, for example, involve administering the antibiotic tetracycline, to activate gene expression via its effect on the tet repressor/VP 16 fusion protein.

The invention will now be further described in the Examples which follow, which are intended as an illustration only and do not limit the scope of the invention. The Examples refer to the Figures.

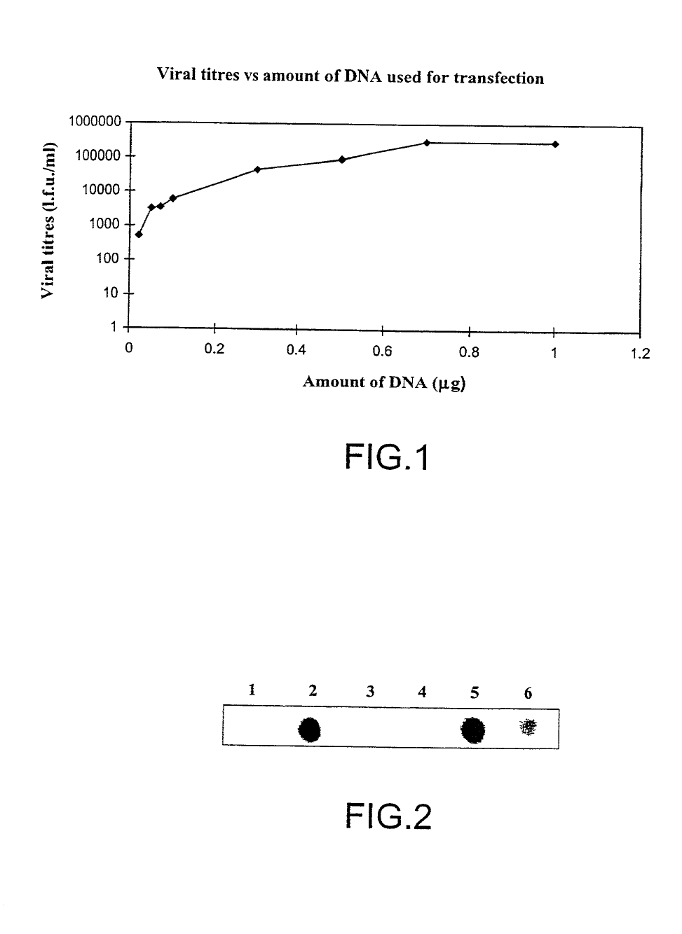

FIG. 1—Graph of viral titres vs amount of DNA encoding viral components

FIG. 2—Reverse transcriptase assays of viral stocks prepared from different amounts of the three components.

Spots: 1, 0.1 μg of all three plasmids; 2. 1 μg of pHIT60, 0.1 μg of pHIT111 and pHIT456; 3, 1 μg of pHIT 111, 0.1 μg of pHIT60 and pHIT456; 4, 1 μg of pHIT456, 0.1 μg of pHIT60 and pHIT111; 5, 1 μg of all three plasmids; 6, 0.1 μg of pHIT60. 1 μg of pHIT 111 and pHIT456. Sample 6 has similar titres to sample 5 although its reverse transcriptase activity is lower.

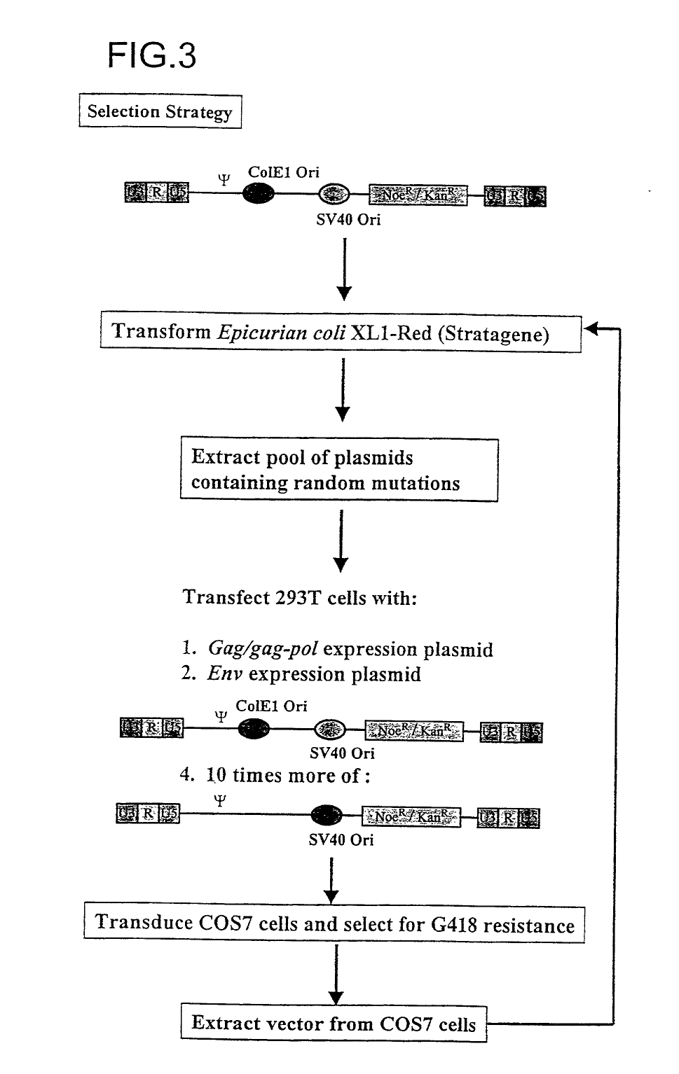

FIG. 3—In vivo selection of a vector with higher packaging efficiency.

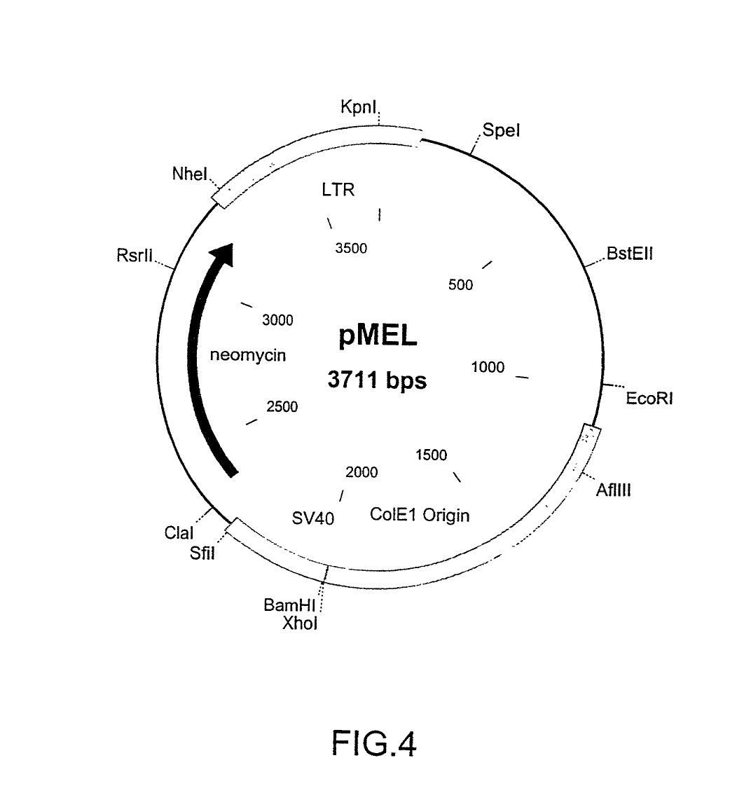

FIG. 4—Map of shuttle vector pMEL.

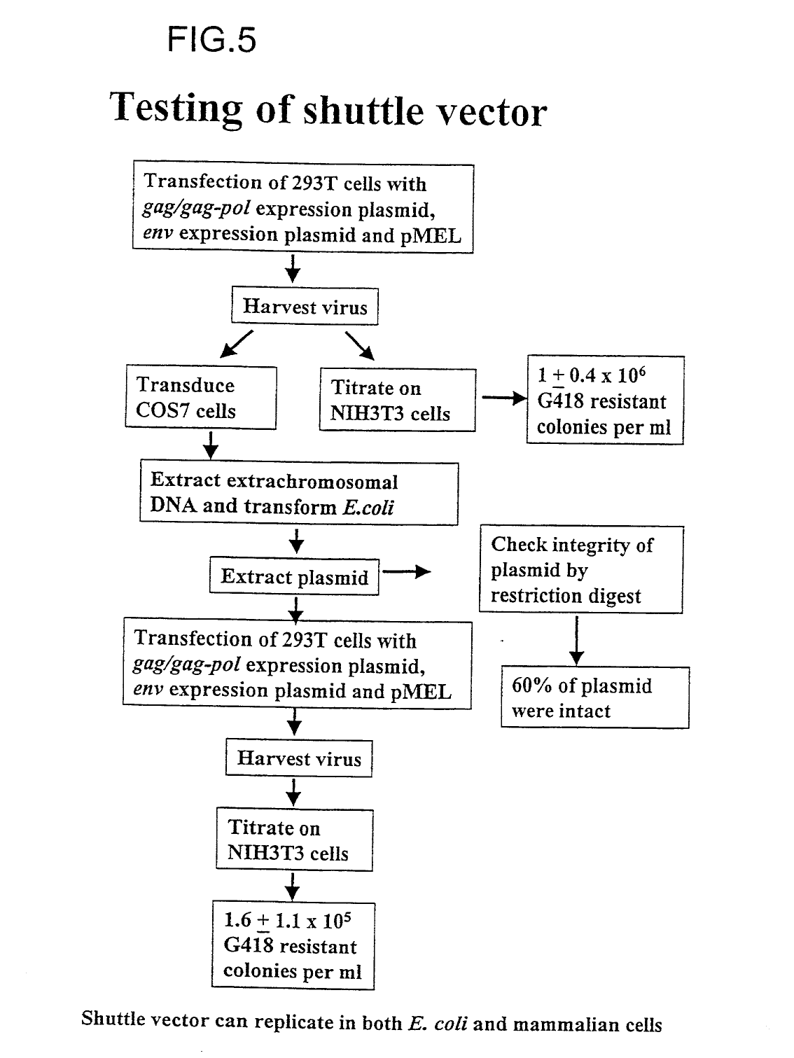

FIG. 5—Diagrammatic representation of scheme for testing shuttle vector

### EXAMPLES

### Example 1

### Effect of Each Viral Component on Viral Titres

We have investigated effects of the stoichiometry of viral components on retroviral production using a transient transfection system. The murine leukaemia virus (MLV) genome was segregated into three different plasmids: one containing the gag/gag-pol, one containing the env and another containing the long terminal repeats, packaging signal and the lacZ marker (genome construct).

Firstly, we determined the conditions under which none of the three viral components are saturating. The results shown in FIG. 1 indicate that since none of the viral components are saturating at 0.1 μg of each plasmid, then 0.1 μg would be a suitable starting point from which the amount of each component could then be raised.

Raising the amount of one plasmid with respect to the other two, we then measured the viral titres and compared them to the titres produced when equal amounts of all three plasmids were used.

The number of infectious particles was determined by X-gal staining of transduced NIH3T3 cells, while the total number of viral particles was measured by reverse transcriptase assay.

The results shown in Table 1 indicate that genome is limiting and that 10-fold more genome increased titres. It was also found that similar titres could be achieved using 10 times less of the gag/gag-pol component compared to using equal amounts of all three components (Table 1). However, the former viral stock had a lower reverse transcriptase activity compared to the latter (FIG. 2—lanes 5 and 6), suggesting that the viral stock produced using less gag/gag-pol contained a larger infectious-particles-to-total-particles ratio. These results showed that it is possible to produce a viral stock in which all the particles are infectious by ensuring that they are all filled with genomic RNA.

TABLE 1
Effect of each component on viral titres.

| Amounts of plasmids used | | | |
|---|---|---|---|
| in transfection (μg)^{a} | | | |
| pHIT60 | pHIT111 | pHIT456 | |
| (gag/gag-pol | (genome) | (env) | Titres (I.f.u./ml)^{b} |
| 0.1 | 0.1 | 0.1 | 6.5 ± 0.9 × 10³ |
| 1 | 0.1 | 0.1 | 1.6 ± 0 × 10³ |
| 0.1 | 1 | 0.1 | 4.1 ± 0.1 × 10⁴ |
| 0.1 | 0.1 | 1 | 1.9 ± 0.4 × 10⁴ |
| 1 | 1 | 1 | 3.5 ± 0.5 × 10⁵ |
| 0.1 | 1 | 1 | 1.6 ± 0.6 × 10⁵ |

^{a}Different amounts of plasmids were used to transfect 293T cells in 6 cm dishes using FuGene6 transfection reagent (Boebringer Mannheim).
^{b}Viral titres were measured as the number of lacZ forming units (l.f.u.) per ml as observed by X-gal staining.

### Example 2

### In Vivo Strategy to Select a Vector Genome with Improved Packaging Efficiency.

We have also developed an in vivo strategy to select a vector genome with improved packaging efficiency which involves shuttling a retroviral vector between a mutagenic strain of Epicurian coli, where random mutations are introduced in its sequence, and mammalian cells, where selection is made for better packaging efficiency (FIG. 3). Selection is effected by competition for packaging by vectors containing the existing packaging signal.

A shuttle retroviral vector is constructed by cloning the bacterial ColE1 origin of replication into the multiple cloning site of pLXSN. To create a vector that confers kanamycin resistance in bacteria a bacterial promoter is inserted upstream of the neomycin resistance gene by replacing the Sfi I-Rsr II fragment with the Sfi I-Rsr II fragment from pEGFPN1 (Clontech). The resulting vector can replicate in E. coli cells under kanamycin selection. When transduced into mammalian cells expressing the SV40 large-T antigen, it replicates extrachromosomally under neomycin selection. The Mammalian cell—E. coli-LTR containing vector is designated pMEL (FIG. 4) and has the following features:

(1) Kanamycin/Neomycin resistance: allows for selection on kanamycin in E. coli and G418 in eucaryotic cells.

(2) ColE1 Origin of replication: allows for high copy number replication in E. coli

(3) SV40 Origin of replication: allows for extrachromosomal replication in eukaryotic cells expressing SV40 large T antigen e.g. COS7 cells (Cepko et al., 1984)

pMEL is transformed into Epicurian Coli XL1-Red cells (Stratagene) to introduce random mutations in its sequence. The pool of mutants is extracted using standard large-scale plasmid isolation protocols.

A competition for packaging is set up by co-transfecting 293T cells with equal amounts of the gag-pol expression plasmid, env expression plasmid, Neo-minus pMEL and ten times less of mutagenised pMEL. Vector genomes that are packaged are used to transduce COS cells. They are then isolated from neomycin resistant cells and used to transform XL1-Red cells. The process is repeated to obtain vector genomes with higher packaging efficiencies.

TABLE 2

| | | | | | Titres |
|---|---|---|---|---|---|
| Amount of plasmid used for transfection (μg) | | | | | (G418 resistant |
| Gag/gag-pol | env | pMEL | pLXSCD8^{a} | pSA91^{b} | colonies per ml) |
| 1 | 1 | 0.1 | 1 | — | 2.0 ± 1.5 × 10³ |
| 1 | 1 | 0.1 | — | 1 | 1.0 ± 0.5 × 10⁴ |

^{a}pLXSN derived vector containing CD8 marker in place of neomycin resistance gene.
^{b}Mammalian expression vector similar in size to pLXSCD8.

An initial round of selection with the shuttle vector gave the results shown above in Table2.

Thus, competition of pMEL for packaging was successfully set up as observed by the decrease in titres in the presence pLXSCD8. Vector genomes emerging from this selection and competition process are packaged more efficiently. The new high efficiency packaging sites can be engineered into any vector genome of the same viral origin using standard recombinant DNA procedures to produce vector systems generating higher titres.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

References

Adam, M. A. and A. D. Miller (1988). “Identification of a signal in a murine retrovirus that is sufficient for packaging of nonretroviral RNA into virions.” J Virol 62(10): 3802-6.

Allen, P., B. Collins, D. Brown, Z. Hostomsky and L. Gold (1996). “A specific RNA structural motiff mediates high affinity binding by the HIV-1 nucleocapsid protein (NCp7).” Virology 225: 306-315.

Beaudry, A. A. and G. F. Joyce (1992). “Directed evolution of an RNA enzyme.” Science 257: 635-641.

Berglund. J. A., B. Charpentier and M. Rosbash (1997). “A high affinity binding site for the HIV-nucleocapsid protein.” Nucleic acids research 25(5): 1042-1049.

Bornscheuer, U. T., M. M. Enzelberger, J. Altenbuchner and H. H. Meyer (1998). “Using XL1-Red mutator strain to generate esterase variants.” Strategies 11(1): 16-17.

Cepko, C. L., B. E. Roberts and R. C. Mulligan. (1984). Cell. 37(3): 1053-62

Coffin et al “Retroviruses” 1997 Cold Spring Harbour Laboratory Press Eds: J M Coffin, S M Hughes, H E Varmus pp 758-763.

Joyce, G. F. (1992). “Directed Molecular Evolution.” Scientific American December: 48-55.

Miller, A. D. (1997). Development and applications of retroviral vectors. Retroviruses. J. M. Coffin, S. H. Hughes and H. E. Varmus, Cold Spring Harbor Press: 437-473.

Soneoka, Y., P. M. Cannon, E. E. Ramsdale, J. C. Griffiths, G. Romano. S. M. Kingsman and A. J. Kingsman (1995). “A transient three-plasmid expression system for the production of high titer retroviral vectors.” Nucleic Acids Res 23(4): 628-33.

Sonstegard, T. S. and P. B. Hackett (1996). “Autogenous regulation of RNA translation and packaging by Rous Sarcoma virus Pr76gag.” J Virol. 70: 6642-6652.

Stemmer. W. P. C. (1994). “Rapid evolution of a protein in vitro by DNA shuffling.” Nature 370: 389-391.

Taplitz, R. A. and J. M. Coffin (1997). “Selection of an avian retrovirus mutant with extended receptor usage.” J Virol 71(10): 7814-9.

Then invention will now be further described by the following numbered paragraphs:

1. A method for selecting an improved retroviral genome having an improved packaging efficiency which method comprises:

(a) introducing one or more random mutations into a retroviral genome comprising a packaging signal;

(b) introducing the mutagenised retroviral genome into a host cell expressing viral polypeptides required for packaging the retroviral genome;

(c) determining whether retroviral packaging efficiency in the cell is improved as compared with a retroviral genome comprising a non-mutated packaging signal;

(d) selecting a viral genome which has improved packaging efficiency.

2. A method according to paragraph 1 wherein an additional step (e) of determining the sequence of all or part of the viral genome to identify the sequence of the packaging signal.

3. A method according to paragraph 1 or 2 wherein step (a) is carried out in a bacterial strain which introduces random mutations into the retroviral genome.

4. A method according to paragraph 3 wherein the bacterial strain is a mutagenic strain of Epicurian coli.

5. A method according to any one of the preceding paragraphs wherein the host cell is a mammalian cell.

6. A method according to any one of the preceding paragraphs wherein the host cell comprises at least:

(i) a first nucleotide sequence encoding a retroviral gag-pol polypeptide;

(ii) a second nucleotide sequence encoding a retroviral envelope polypeptide; and

(iii) a third nucleotide encoding a retroviral genome comprising a non-mutated packaging signal,

wherein the third nucleotide is present as part of a nucleic acid vector which lacks a selectable marker; the mutagenised retroviral genome is present as part of a nucleic acid vector which contains the selectable marker; and the ratio of the vector comprising the third nucleotide to the vector comprising the mutagenised retroviral genome is greater than 2:1, preferably greater than 5:1.

7. A method according to any one of the preceding paragraphs wherein the packaging efficiency, measured as the proportion of the number of infectious retroviral particles to the total number of retroviral particles produced is greater than 25%.

8. A retroviral genome obtained by the method of any one of the preceding paragraphs.

9. A retroviral genome according to paragraph 8 having a packaging efficiency, measured as the proportion of the number of infectious retroviral particles to the total number of retroviral particles produced is greater than 25%.

10. A retroviral packaging signal obtainable from a retroviral genome according to paragraph 8 or 9.

11. A nucleic acid comprising a retroviral packing signal according to paragraph 10.

12. A retroviral vector comprising a retroviral packing signal according to paragraph 10.

13. A retroviral vector according to paragraph 12 for use in producing infectious retroviral particles.

14. A retroviral vector according to paragraph 12 or 13, which is a lentiviral vector.

15. A producer cell comprising a retroviral genome according to paragraph 8 or 9, a retroviral packaging signal according to paragraph 10, or a retroviral vector according to paragraph 12, 13 or 14.

16. A method for enhancing the efficiency of retroviral packaging which method comprises expressing in a producer cell at least a first nucleotide sequence encoding a retroviral gag-pol polypeptide, a second nucleotide sequence encoding a retroviral envelope polypeptide and a third nucleotide sequence encoding a retroviral genome wherein the ratio of the first nucleotide sequence to the second nucleotide sequence and the ratio of the first nucleotide sequence to the third nucleotide sequence is x:y and x:z, respectively wherein x is less than 1 and y and z are 1.

17. A method according to paragraph 16 wherein x is less than 0.5.

18. A method according to paragraph 16 or 17 wherein the packaging efficiency, measured as the proportion of the number of infectious retroviral particles to the total number of retroviral particles produced is greater than 25%.

19. A composition comprising infectious retroviral particles produced according to the method of any one of paragraphs 16 to 18.

20. A composition according to paragraph 19 for use in therapy.

21. A producer cell which expresses at least a first nucleotide sequence encoding a retroviral gag-pol polypeptide, a second nucleotide sequence encoding a retroviral envelope polypeptide and a third nucleotide sequence encoding a retroviral genome wherein the ratio of the first nucleotide sequence to the second nucleotide sequence and the ratio of the first nucleotide sequence to the third nucleotide sequence is x:y and x:z, respectively wherein x is less than 1 and y and z are 1.

1. A method for selecting an improved retroviral genome having an improved packaging efficiency which method comprises:
(a) introducing one or more random mutations into a retroviral genome comprising a packaging signal;
(b) introducing the mutagenised retroviral genome into a host cell expressing viral polypeptides required for packaging the retroviral genome;
(c) determining whether retroviral packaging efficiency in the cell is improved as compared with a retroviral genome comprising a non-mutated packaging signal;
(d) selecting a viral genome which has improved packaging efficiency.

2. A method according to claim 1 wherein an additional step (e) of determining the sequence of all or part of the viral genome to identify the sequence of the packaging signal.

3. A method according to claim 1 wherein step (a) is carried out in a bacterial strain which introduces random mutations into the retroviral genome.

4. A method according to claim 3 wherein the bacterial strain is a mutagenic strain of Epicurian coli.

5. A method according to claim 1 wherein the host cell is a mammalian cell.

6. A method according to claim 1 wherein the host cell comprises at least:
(i) a first nucleotide sequence encoding a retroviral gag-pol polypeptide;
(ii) a second nucleotide sequence encoding a retroviral envelope polypeptide; and
(iii) a third nucleotide encoding a retroviral genome comprising a non-mutated packaging signal,
wherein the third nucleotide is present as part of a nucleic acid vector which lacks a selectable marker; the mutagenised retroviral genome is present as part of a nucleic acid vector which contains the selectable marker; and the ratio of the vector comprising the third nucleotide to the vector comprising the mutagenised retroviral genome is greater than 2:1, preferably greater than 5:1.

7. A method according to claim 1 wherein the packaging efficiency, measured as the proportion of the number of infectious retroviral particles to the total number of retroviral particles produced is greater than 25%.

8. A retroviral genome obtained by the method of claim 1.

9. A retroviral genome according to claim 8 having a packaging efficiency, measured as the proportion of the number of infectious retroviral particles to the total number of retroviral particles produced is greater than 25%.

10. A retroviral packaging signal obtainable from a retroviral genome according to claim 8.

11. A nucleic acid comprising a retroviral packing signal according to claim 10.

12. A retroviral vector comprising a retroviral packing signal according to claim 10.

13. A retroviral vector according to claim 12 for use in producing infectious retroviral particles.

14. A retroviral vector according to claim 12, which is a lentiviral vector.

15. A producer cell comprising a retroviral genome according to claim 8, a retroviral packaging signal obtainable from a retroviral genome, or a retroviral vector comprising a retroviral packing signal obtainable from a retroviral genome, or a retroviral vector comprising a retroviral packing signal obtainable from a retroviral genome for use in producing infectious retroviral particles or a retroviral vector comprising a retroviral packing signal obtainable from a retroviral genome for use in producing infectious retroviral particles wherein the retroviral vector is a lentiviral vector.

16. A method for enhancing the efficiency of retroviral packaging which method comprises expressing in a producer cell at least a first nucleotide sequence encoding a retroviral gag-pol polypeptide, a second nucleotide sequence encoding a retroviral envelope polypeptide and a third nucleotide sequence encoding a retroviral genome wherein the ratio of the first nucleotide sequence to the second nucleotide sequence and the ratio of the first nucleotide sequence to the third nucleotide sequence is x:y and x:z, respectively wherein x is less than 1 and y and z are 1.

17. A method according to claim 16 wherein x is less than 0.5.

18. A method according to claim 16 wherein the packaging efficiency, measured as the proportion of the number of infectious retroviral particles to the total number of retroviral particles produced is greater than 25%.

19. A composition comprising infectious retroviral particles produced according to the method of claim 16.

20. A composition according to claim 19 for use in therapy.

21. A producer cell which expresses at least a first nucleotide sequence encoding a retroviral gag-pol polypeptide, a second nucleotide sequence encoding a retroviral envelope polypeptide and a third nucleotide sequence encoding a retroviral genome wherein the ratio of the first nucleotide sequence to the second nucleotide sequence and the ratio of the first nucleotide sequence to the third nucleotide sequence is x:y and x:z, respectively wherein x is less than 1 and y and z are 1.
